SEQUENCE LISTING (9 sequences)

SEQ ID NO: 1 - protein, 349 aa, Homo sapiens
MTIGEMLRSFLTKLEWFSTLFPRIPVPVQKNIDQQIKTRPRKIKKDGKEGAEEIDRHVERRRSRSPRRSLSPRRSPRRSRSRSHHREGHGSSSFDRELEREKERQRLEREAKEREKERRRSRSIDRGLERRRSRSRERHRSRSRSRDRKGDRRDRDREREKENERGRRRDRDYDKERGNEREKERERSRERSKEQRSRGEVEEKKHKEDKDDRRHRDDKRDSKKEKKHSRSRSRERKHRSRSRSRNAGKRSRSRSKEKSSKHKNESKEKSNKRSRSGSQGRTDSVEKSKKREHSPSKEKSRKRSRSKERSHKRDHSDSKDQSDKHDRRRSQSIEQESQEKQHKNKDETV

SEQ ID NO: 2 - DNA, 2062 bp, Homo sapiens
  misc_feature (2037)..(2037): n is a, c, g, or t
acgcggggtttccaattatttgttcatttatttattttctacataactaaattagaaacctcactgcttcatggcagttggtttgctattgcttccagttttattagggcttcattttatattagagctgttaaaagataacctttagacaggaattatctaaagtagacattttatattagagctgttaaaagataacctttagacaggaattatctaaagtagatcatatgtagctaggttatggtgcaaggtgtatgatgtgtgcaaatatgtccacagaaataaatacatagtaggtatgtggaatgtaaatttaagtcaatcgttccgcatagtttagaaatgtaaggggctttttcatattgttaactgagtgagatcagttccctttatgcctgtgaggctgcagggtttgttctcacttgcatgcacacactaagcccaaatatttctgttcattcattgtcagatcaggatatgaaaataaaatttttctgttagttttttttgtattgagattccaaagatggtaatatttttataatattcatgtatatatggaaatactttttttgacggctagggtatcttttgtgtttctgtaggacctagatgtgaaggctggtggaggctgtgtaatgaccattggagaaatgctacgatcttttctcacaaaactggagtggttttctaccttgtttccaagaattccagttccagttcaaaagaatattgatcaacagattaaaacccgacctagaaaaatcaagaaagatgggaaggaaggtgctgaggaaatagacagacatgttgaacgcagacgttcaaggtctccaaggagatctctgagtccacggaggtccccaagaaggtcaagaagtagaagtcatcatcgggagggccatgggtcttctagttttgacagagaattagaaagagagaaagaacgccagcgactagagcgtgaagccaaagaaagggagaaagaacggcgaagatcccgaagtattgaccgggggttagaacgcaggcgcagcagaagtagggaaaggcatagaagtcgcagtcgaagtcgtgataggaaaggggatagaagggacagggatcgagaaagagagaaagaaaatgagagaggtagaagacgagatcgtgactatgataaggaaagaggaaatgaacgagaaaaagagagagagcgatcaagagaaaggtccaaggaacagagaagtaggggagaggtagaagagaagaaacataaagaagacaaagatgataggcggcacagagatgacaaaagagattccaagaaagagaaaaaacacagtagaagcagaagcagagaaaggaaacacagaagtaggagtcgaagtagaaatgcagggaaacgaagtagaagtagaagcaaagagaaatcaagtaaacataaaaatgaaagtaaagaaaaatcaaataaacgaagtcgaagtggcagtcaaggaagaactgacagtgttgaaaaatcaaaaaaacgggaacatagtcccagcaaagaaaaatctagaaagcgtagtagaagcaaagaacgttcccacaaacgagatcacagtgatagtaaggaccagtcagacaaacatgatcgtcgaaggagccaaagtatagaacaagagagccaagaaaaacagcataaaaacaaagatgagactgtgtgaaaatattttgtaaaagtggatcacattgaatcctataaatgattaaatctgcttttttcccccacgttgagattgtgcagtagttcgcactcctcaagctctccctgtaggctgcattttcatttcctctttcgtgtagggaagtgcctttgtaattccatttattgcattggtgttttcacccaattgttaagtttgatacatgatgcacagattgttcttgcatttttattgtttgtttttgaaatgtacagtctgtacatatgtcctgaaaatgttttaattcctttggcatggttaccatgttggttaaatttgtataaggcaataaactgccactaatccnaaaaaaaaaaaaaaaaaaaaaaaaa

SEQ ID NO: 3 - DNA, 1050 bp, Homo sapiens
atgaccattggagaaatgctacgatcttttctcacaaaactggagtggttttctaccttgtttccaagaattccagttccagttcaaaagaatattgatcaacagattaaaacccgacctagaaaaatcaagaaagatgggaaggaaggtgctgaggaaatagacagacatgttgaacgcagacgttcaaggtctccaaggagatctctgagtccacggaggtccccaagaaggtcaagaagtagaagtcatcatcgggagggccatgggtcttctagttttgacagagaattagaaagagagaaagaacgccagcgactagagcgtgaagccaaagaaagggagaaagaacggcgaagatcccgaagtattgaccgggggttagaacgcaggcgcagcagaagtagggaaaggcatagaagtcgcagtcgaagtcgtgataggaaaggggatagaagggacagggatcgagaaagagagaaagaaaatgagagaggtagaagacgagatcgtgactatgataaggaaagaggaaatgaacgagaaaaagagagagagcgatcaagagaaaggtccaaggaacagagaagtaggggagaggtagaagagaagaaacataaagaagacaaagatgataggcggcacagagatgacaaaagagattccaagaaagagaaaaaacacagtagaagcagaagcagagaaaggaaacacagaagtaggagtcgaagtagaaatgcagggaaacgaagtagaagtagaagcaaagagaaatcaagtaaacataaaaatgaaagtaaagaaaaatcaaataaacgaagtcgaagtggcagtcaaggaagaactgacagtgttgaaaaatcaaaaaaacgggaacatagtcccagcaaagaaaaatctagaaagcgtagtagaagcaaagaacgttcccacaaacgagatcacagtgatagtaaggaccagtcagacaaacatgatcgtcgaaggagccaaagtatagaacaagagagccaagaaaaacagcataaaaacaaagatgagactgtgtga

SEQ ID NO: 4 - DNA, 25 bp, Homo sapiens
cggcgaagatcccgaagtattgacc

SEQ ID NO: 5 - DNA, 22 bp, Artificial
  T128 Primer 1 (Synthetic oligonucleotide primer)
gagagagcgatcaagagaaagg

SEQ ID NO: 6 - DNA, 20 bp, Artificial
  T128 Primer 2 (Synthetic oligonucleotide primer)
atctctgtgccgcctatcat

SEQ ID NO: 7 - DNA, 28 bp, Artificial
  5′ RACE gene specific primer (Synthetic oligonucleotide primer)
tcttccttgactgccacttcgacttcgt

SEQ ID NO: 8 - DNA, 25 bp, Artificial
  3′ RACE gene specific primer (Synthetic oligonucleotide primer)
cggccaagatcccgaagtattgacc

SEQ ID NO: 9 - DNA, 28 bp, Artificial
  3′ RACE gene specific nested primer (Synthetic oligonucleotide primer)
acgaagtcgaagtggcagtcaaggaaga

(19) United States
(12) Patent Application Publication
(10) Pub. No.: US 2007/0286862 A1
(43) Pub. Date: Dec. 13, 2007

(54) Cancer Associated Antigens

(76) Inventors: Robert Charles Rees, Bradway (GB); Amanda Kathleen Miles, Heanor (GB); Geng Li, Beeston (GB)

Correspondence Address: BARNES & THORNBURG LLP, 11 SOUTH MERIDIAN, INDIANAPOLIS, IN, 46204, US

(21) Appl. No.: 10/594,791

(22) PCT Filed: Mar. 24, 2005
(86) PCT No.: PCT/GB05/01197

§ 371 Date: Jul. 18, 2007

(30) Foreign Application Priority Data
Apr. 2, 2004 (GB) 0407587.5

(51) Int. Cl. A61K 31/70; A61K 38/00; A61K 39/00; A61P 43/00; C07H 21/04; C07K 14/00; C12N 15/00; C12N 5/00; C12P 21/08; C12Q 1/68; G01N 33/53

(52) U.S. Cl. 424/142.1; 424/277.1; 435/320.1; 435/325; 435/6; 435/7.23; 514/2; 514/44; 530/350; 530/388.15; 536/23.5

(57) ABSTRACT

The invention relates to isolated nucleic acid sequences which are expressed in cancers, especially gastrointestinal, kidney and prostate cancers, to their protein products and to the use of nucleic acid and protein products for the identification and treatment of cancers.

The invention relates to isolated nucleic acid sequences which are expressed in cancers, especially gastrointestinal, kidney and prostate cancers, to their protein products and to the use of the nucleic acid and protein products for the identification and treatment of cancers.

Cancers of the intestinal tract, such as gastric carcinomas and colorectal cancers, account for up to 15% of cancer-related deaths in the United States, and have low survival rates. Such cancers are often asymptomatic, the patient only becoming aware of them when the cancers have progressed too far to be successfully treated. There is therefore a need to identify new diagnostic tools and methods for treating such cancers.

The prostate gland is an accessory sex gland in males which is wrapped around the urethra as this tube leaves the bladder. The gland secretes an alkaline fluid during ejaculation. Cancer of the prostate gland is very serious and represents the second leading cause of death from cancer in men.

Two specific proteins are known to be made in very high concentrations in prostate cancer cells. These are prostatic acid phosphatase (PAP) and prostate specific antigen (PSA). These proteins have been characterised and have been used to follow response to therapy. However, it has been difficult to correlate the presence of these two proteins to the presence of cancer.

Accordingly, there is a need to identify new genes and proteins which are associated with the presence of prostate cancer.

Identification of immunogenic proteins in cancer is essential for the development of immunotherapeutic strategies where adoptive immunity is directed towards MHC Class I- and Class II-associated peptides (Mians, et al., Cancer Immunology (2001), page 1). Many antigens are implicated in aetiology and progression of cancer, and are associated with epigenetic events. Pre-clinical and clinical studies infer that vaccination and targeting MHC-associated peptide antigens promotes tumour rejection (Ali S. A., et al, J Immunol. (2002), Vol. 168(7), pages 3512-19 and Rees R. C., et al., Immunol. Immunother (2002), Vol 51(1), pages 58-61).

The inventors have used a technique known as SEREX (Serological analysis of recombinant cDNA expression libraries) to identify genes which are over-expressed in cancer tissue. This technique was published by Sahin et al (PNAS (USA), 1995, Vol. 92, pages 11810-11813). SEREX normally uses total RNA isolated from tumour biopsies from which poly(A)⁺ RNA is then isolated. cDNA is then produced using an oligo (dT) primer. The cDNA fragments produced are then cloned into a suitable expression vector, such as a bacteriophage and cloned into a suitable host, such as E. coli. The clones produced are screened with high-titer IgG antibodies in autologous patient serum, to identify antigens associated with the tumour.

Several SEREX-defined antigens nave provided attractive candidates for the construction of cancer vaccines, for example NY-ESO-1 from testis (Chen Y. T., et al. (1997), Vol. 4, page 1914; Stockert E., et al., J. Exp. Med. (1998), Vol. 187, page 1349; Jager E., et al. PNAS (2000), Vol. 97, page 12198; and Jager E., et al., PNAS (2000), Vol. 97, page 4760). Mutated p53 (Scanlan M. J., et al., Int. J. Cancer (1998), Vol. 76, page 652), putative tumour suppressor ING 1 (Jager D. et al., Cancer Res. (1999), Vol. 59, page 6416) and adhesion molecule galectin 9 (Tureci O., et al., J. Biol. Chem. (1997), Vol. 272, page 6416), for example, have been detected by SEREX, showing that the analysis of autoantibodies can identify genes involved in cancer etiology and identify diagnostic markers or indicators of disease progression.

The inventors unexpectedly realised that some cancers express antigens that are also found in normal testes tissues. They therefore adapted the SEREX technique to screen a normal testicular tissue cDNA library against serum from pooled allogenic prostate cancer patients. This unexpectedly identified an antigen (“T128”) which is highly expressed in malignant tissues, especially prostate and gastric cancers.

The testes cDNA library was screened using pooled allogeneic prostate cancer patients' sera. Seven reactive clones were purified, in vivo excised, and converted to plasmid forms. cDNA inserts were analysed using restriction mapping and cDNA sequencing. Comparison to the Genbank non-redundant and expressed sequence tag (EST) databases revealed that these 7 clones represented 6 distinct genes, 5 previously unknown genes and 1 known gene. T128 was fully sequenced and is described here; the following results are consistent with T128 being a cancer-testis (CT) associated gene, the predicted protein of which it is believed will act as a new target antigen for immunotherapy.

T128 overlaps with a predicted, hypothetical coding sequence submitted to Genbank as accession number FLJ10330. The function and isolation of this was not referred to. This hypothetical sequence was apparently found by Lee S. Y., et al. (PNAS, (2003), Vol. 100(5), pages 2651-2656) to be similar to an antigen found by serological analysis of cDNA expression libraries (clone NY-SAR-27). However, information published on this clone is limited and only seems to refer to a partial CDS. T128 is the full CDS. Furthermore, NY-SAR-27 is only mentioned in passing by Lee and was not developed further, presumably due to the limited reactivity observed (only 2 out of 39 sarcoma sera). This is in contrast with the full sequence now identified by the inventors and unexpectedly found to have considerably greater potential.

The invention provides an isolated mammalian nucleic acid molecule selected from the group consisting of:

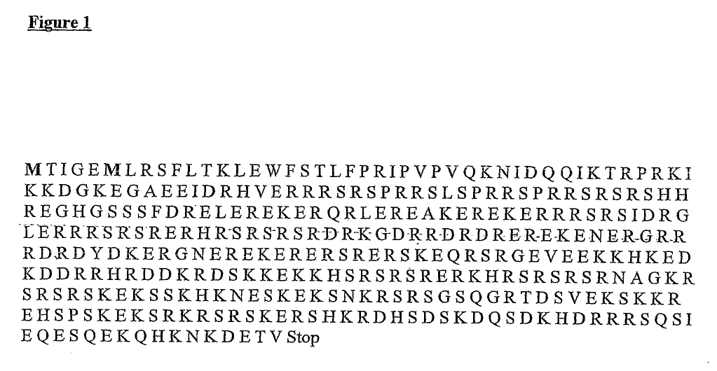

(a) Nucleic acid molecules encoding T128 polypeptide, as shown in FIG. 1, a polypeptide at least 80% identical to T128, or a fragment thereof which is capable of cross-reacting with sera from patients with prostate cancer.

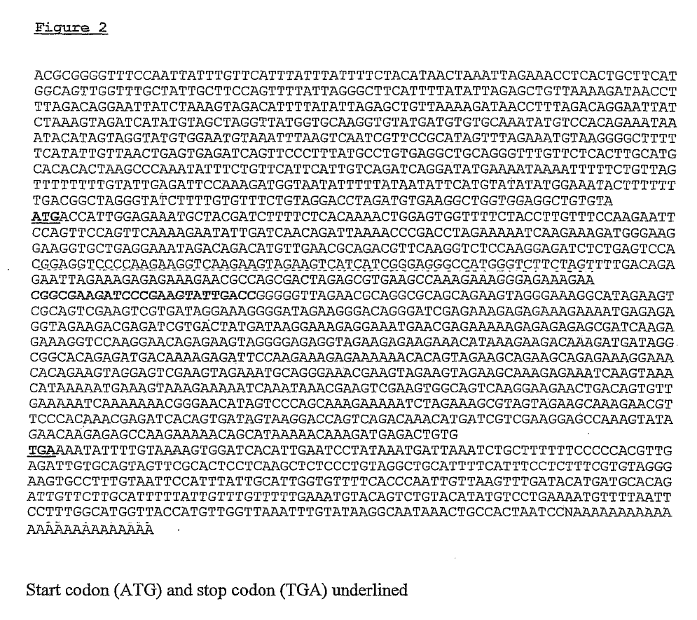

(b) Nucleic acid molecules comprising the nucleotide sequence depicted in SEQ. ID. 1 between nucleic acid residues the start (ATG) and stop codon (TGA) of FIG. 2 (i.e. 642 to 1688), also shown in SEQ. ID No. 3.

(c) Nucleic acid molecules, the complementary strand of which specifically hybridises to a nucleic acid molecule in (a) or (b).

(d) Nucleic acid molecules the sequence of which differs from the sequence of the nucleic acid molecule of (C) due to the degeneracy of the genetic code.

Preferably the nucleic acid molecule encodes T128. Preferably, the polypeptide is expressed in higher than normal concentrations in gastric, kidney and/or prostate cancer tissue, compared to one or more of normal lung, liver, kidney, heart, brain, trachea, and/or prostate.

Preferably the polypeptide is expressed in higher concentrations of normal testicular tissue compared with the normal tissues listed above.

Preferably the nucleic acid molecule encodes a polypeptide which is capable of acting as a transcription factor. That is, it is capable of binding a DNA molecule and regulating the transcription of a region of that DNA molecule by an RNA polymerase. Most preferably the polypeptide has nuclear localisation. Initial analysis of the data shows that this is a property of the polypeptide.

The sequence especially has one or more of the following features:

Chromosomal localization Ip13.3

N-glycosylation site at position 264-267

cAMP-phosphorylation sites 60-63, 118-121, 130-133, 219-222, 226-229, 301-304, 327-330.

PKC phosphorylation sites at positions 65-67, 71-73, 75-77, 222-224, 259-261, 270-272, 288-290, 300-302, 310-312, 322-324.

Casein kinase 2 phosphorylation sites at positions 2-5, 12-15, 92-95, 197-200, 222-225.

Tyrosine phosphorylation sites at position 167-173.

Amidation sites at positions 165-169, 247-250.

Arginine rich region 56-254.

Lysine rich region 204-322.

Senine rich region 229-322.

Nucleic acid molecules having at least 60%, 70%, 75%, 80%, 85%, 90%, 95%, 98%, 99% homology to the nucleic acid molecules are also provided. Preferably these express proteins which are expressed in higher concentrations in cancerous tissue than the equivalent normal tissue. That is they are higher in e.g. prostate cancer or gastric cancers than normal, non-cancerous, prostate, kidney or normal non-cancerous gastric tissue. The proteins are preferably expressed in higher concentrations in gastric, kidney and/or prostate cancer tissue, more preferably and normal testes than in e.g. one or more of other, normal tissues such as normal lung, liver, heart, brain, trachea, and/or prostate. Preferably this is at least 2, most preferably at least 5 times higher concentrations than normal tissue.

The nucleic acid molecules of the invention may be DNA, cDNA or RNA. In RNA molecules “T” (Thymine) residues may be replaced by “U” (Uridine) residues.

Preferably, the isolated mammalian nucleic acid molecule is an isolated human nucleic acid molecule.

The invention further provides nucleic acid molecules comprising at least 15 nucleotides capable of specifically hybridising to a sequence included within the sequence of a nucleic acid molecule according to the first aspect of the invention. The hybridising nucleic acid molecule may either be DNA or RNA. Preferably the molecule is at least 90%, at least 92%, at least 94%, at least 96%, at least 98%, at least 99%, homologous to the nucleic acid molecule according to the first aspect of the invention. This may be determined by techniques known in the art.

The term “specifically hybridising” is intended to mean that the nucleic acid molecule can hybridise to nucleic acid molecules according to the invention under conditions of high stringency. Typical conditions for high stringency include 0.1×SET, 0.1% SDS at 68° C. for 20 minutes.

The invention also encompasses variant nucleic acid molecules such as DNAs and cDNAs which differ from the sequences identified above, but encode the same amino acid sequences as the isolated mammalian nucleic acid molecules, by virtue of redundancy in the genetic code.

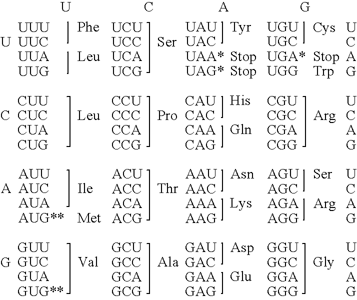

*Chain-terminating, or “nonsense” codons.
**Also used to specify the initiator formyl-Met-tRNAMet. The Val triplet GUG is therefore “ambiguous” in that it codes both valine and methionine.

The Genetic Code Showing mRNA Triplets and the Amino Acids for Which They Code

The invention also includes within its scope vectors comprising a nucleic acid according to the invention. Such vectors include bacteriophages, phagemids, cosmids and plasmids. Preferably the vectors comprise suitable regulatory sequences, such as promoters and termination sequences which enable the nucleic acid to be expressed upon insertion into a suitable host. Accordingly, the invention also includes hosts comprising such a vector. Preferably the host is E. coli.

A second aspect of the invention provides an isolated polypeptide obtainable from a nucleic acid sequence according to the invention. As indicated above, the genetic code for translating a nucleic acid sequence into an amino acid sequence is well known.

Preferably the sequence comprises the sequence shown in FIG. 1.

The invention further provides polypeptide analogues, fragments or derivatives of antigenic polypeptides which differ from naturally-occurring forms in terms of the identity of location of one or more amino acid residues (deletion analogues containing less than all of the residues specified for the protein, substitution analogues wherein one or more residues specified are replaced by other residues in addition analogues wherein one or more amino acid residues are added to a terminal or medial portion of the polypeptides) and which share some or all properties of the naturally-occurring forms. Preferably such polypeptides comprise between 1 and 20, preferably 1 and 10 amino acid deletions or substitutions.

Preferably the polypeptide is at least 80%, 85%, 90%, 95%, 96%, 97%, 98% or 99% identical to the sequences of the invention. This can be determined conventionally using known computer programs such as the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

Preferably the polypeptides of the invention do not contain one or more amino acid residues from upstream of the sequence shown in FIG. 1 from the sequence shown as FLJ10330. That is one or more amino acids before MTIGEMLR . . . etc. found in sequence FLJ10330.

The nucleic acids and polypeptide of the invention are preferably identifiable using the SEREX method. However, alternative methods, known in the art, may be used to identify nucleic acids and polypeptides of the invention. These include differential display PCR (DD-PCR), representational difference analysis (RDA) and suppression subtracted hybridisation (SSH).

The nucleic acid molecules encoding T128 according to the invention and the polypeptides which they encode are detectable by SEREX (discussed below). The technique uses serum antibodies from cancer patients to identify the molecules. It is therefore the case that the gene products identified by SEREX are able to evoke an immune response in a patient and may be considered as antigens suitable for potentiating further immune reactivity if used as a vaccine.

The third aspect of the invention provides the use of nucleic acids or polypeptides according to the invention, to detect or monitor cancers, preferably gastrointestinal cancers, such as gastric cancer or prostate cancer.

The use of a nucleic acid molecule hybridisable under high stringency conditions, a nucleic acid according to the first aspect of the invention to detect or monitor cancers, e.g. gastro-intestinal cancers, such as gastric cancer or colorectal cancer, or prostate cancer, is also encompassed. Such molecules may be used as probes, e.g. using PCR.

The expression of genes, and detection of their polypeptide products may be used to monitor disease progression during therapy or as a prognostic indicator of the initial disease status of the patient.

There are a number of techniques which may be used to detect the presence of a gene, including the use of Northern blot and reverse transcription polymerase chain reaction (RT-PCR) which may be used on tissue or whole blood samples to detect the presence of cancer associated genes. For polypeptide sequences in-situ staining techniques or enzyme linked ELISA assays or radio-immune assays may be used. RT-PCR based techniques would result in the amplification of messenger RNA of the gene of interest (Sambrook, Fritsch and Maniatis, Molecular Cloning, A Laboratory Manual, 2^{nd} Edition). ELISA based assays necessitate the use of antibodies raised against the protein or peptide sequence and may be used for the detection of antigen in tissue or serum samples (McIntyre C. A., Rees R.C. et. al., Europ. J. Cancer 28, 58-631 (1990)). In-situ detection of antigen in tissue sections also rely on the use of antibodies, for example, immuno peroxidase staining or alkaline phosphatase staining (Gaepel, J. R., Rees, R. C. et. al., Brit. J. Cancer 64, 880-883 (1991)) to demonstrate expression. Similarly radio-immune assays may be developed whereby antibody conjugated to a radioactive isotope such as I¹²⁵ is used to detect antigen in the blood.

Blood or tissue samples may be assayed for elevated concentrations of the nucleic acid molecules or polypeptides.

Preferably elevated levels of the molecules in tissues that are not normal testes is indicative of the presence of cancerous tissues.

Methods of producing antibodies which are specific to the polypeptides of the invention, for example, by the method of Kohler & Milstein to produce monoclonal antibodies, are well know. A further aspect of the invention provides an antibody, e.g. a monoclonal antibody, which specifically binds to a polypeptide according to the invention.

Kits for detecting or monitoring cancer, such as gastro-intestinal cancers, including gastric cancer and/or colorectal cancer, or prostate cancer, using polypeptides, nucleic acids or antibodies according to the invention are also provided. Such kits may additionally contain instructions and reagents to carry out the detection or monitoring.

The fourth aspect of the invention provides for the use of nucleic acid molecules according to the first aspect of the invention or polypeptide molecules according to the second aspect of the invention in the prophylaxis or treatment of cancer, or pharmaceutically effective fragments thereof. By pharmaceutically effective fragment, the inventors mean a fragment of the molecule which still retains the ability to be a prophylactant or to treat cancer. The cancer may be a gastrointestinal cancer, such as gastric cancer or colorectal cancer.

The molecules are preferably administered in a pharmaceutically amount. Preferably the dose is between 1 μg/kg. to 10 mg/kg.

The nucleic acid molecules may be used to form DNA-based vaccines. From the published literature it is apparent that the development of protein, peptide and DNA based vaccines can promote anti-tumour immune responses. In pre-clinical studies, such vaccines effectively induce a delayed type hypersensitivity response (DTH), cytotoxic T-lymphocyte activity (CTL) effective in causing the destruction (death by lysis or apoptosis) of the cancer cell and the induction of protective or therapeutic immunity. In clinical trials peptide-based vaccines have been shown to promote these immune responses in patients and in some instances cause the regression of secondary malignant disease. Antigens expressed in prostate cancer (or other types of cancers) but not in normal tissue (or only weakly expressed in normal tissue compared to cancer tissue) will allow us to assess their efficacy in the treatment of cancer by immunotherapy. Polypeptides derived from the tumour antigen may be administered with or without immunological adjuvant to promote T-cell responses and induce prophylactic and therapeutic immunity. DNA-based vaccines preferably consist of part or all of the genetic sequence of the tumour antigen inserted into an appropriate expression vector which when injected (for example via the intramuscular, subcutaneous or intradermal route) cause the production of protein and subsequently activate the immune system. An alternative approach to therapy is to use antigen presenting cells (for example, dendritic cells, DC's) either mixed with or pulsed with protein or peptides from the tumour antigen, or transfect DC's with the expression plasmid (preferably inserted into a viral vector which would infect cells and deliver the gene into the cell) allowing the expression of protein and the presentation of appropriate peptide sequences to T-lymphocytes or adaptive cellular therapy using, e.g., T-cells responsive to T128 peptides or T128 protein. A DNA based vaccine is demonstrated in, for example, Thompson S. A., et al. (J. Immunol. (1998), Vol. 160, pages 1717-1723).

Accordingly, the invention provides a nucleic acid molecule according to the invention in combination with a pharmaceutically-acceptable carrier.

Such polypeptides may be bound to a carrier molecule such as tetanus toxoid to make the polypeptide immunogenic. Such constructs are also within the scope of the invention.

A further aspect of the invention provides a method of prophylaxis or treatment of a cancer such as a gastro-intestinal cancer, or prostate cancer, comprising the administration to a patient of a nucleic acid molecule according to the invention.

The polypeptide molecules according to the invention may be used to produce vaccines to vaccinate against a cancer, such as a gastrointestinal cancer or prostate cancer.

Accordingly, the invention provides a polypeptide according to the invention in combination with a pharmaceutically acceptable carrier.

The invention further provides use of a polypeptide according to the invention in a prophylaxis or treatment of a cancer such as a gastrointestinal cancer, kidney cancer or prostate cancer.

Methods of prophylaxis or treating a cancer, such as a gastro-intestinal cancer, or prostate cancer, by administering a protein or peptide according to the invention to a patient, are also provided.

Vaccines comprising nucleic acid and/or polypeptides according to the invention are also provided. The polypeptide may be attached to another carrier peptide such as tetanus toxoid to increase the immogenicity of the polypeptide.

The polypeptides of the invention may be used to raise antibodies. In order to produce antibodies to tumour-associated antigens procedures may be used to produce polyclonal antiserum (by injecting protein or peptide material into a suitable host) or monoclonal antibodies (raised using hybridoma technology). In addition PHAGE display antibodies may be produced, this offers an alternative procedure to conventional hybridoma methodology. Having raised antibodies which may be of value in detecting tumour antigen in tissues or cells isolated from tissue or blood, their usefulness as therapeutic reagents could be assessed. Antibodies identified for their specific reactivity with armour antigen may be conjugated either to drugs or to radioisotopes. Upon injection it is anticipated that these antibodies localise at the site of tumour and promote the death of tumour cells through the release of drugs or the conversion of pro-drug to an active metabolite. Alternatively a lethal effect may be delivered by the use of antibodies conjugated to radioisotopes. In the detection of secondary/residual disease, antibody tagged with radioisotope could be used, allowing tumour to be localised and monitored during the course of therapy. Unconjugated antibodies can also be useful in influencing cancer cell growth. For example, the binding of certain antibodies to cell-surface receptors on cancer cells may initiate cell death by, e.g., apoptosis. Therefore the antibodies of this invention could be therapeutically useful in a non-conjugated form.

The term “antibody” includes intact molecules as well as fragments such as Fa, F(ab′)₂ and Fv.

The invention accordingly provides a method of treating a cancer such as gastro-intestinal, kidney cancer, or prostate cancer, by the use of one or more antibodies raised against a polypeptide of the invention.

The cancer-associated proteins identified may form targets for therapy.

The invention also provides nucleic acid probes capable of binding sequences of the invention under high stringency conditions. These may have sequences complementary to the sequences of the invention and may be used to detect mutations identified by the inventors. Such probes may be labeled by techniques known in the art, e.g. with radioactive or fluorescent labels.

Preferably the cancer which is detected, assayed for, monitored, treated or targeted for prophylaxis, is a gastric cancer or prostate cancer.

The invention will now be described by reference to the following figure and examples:

FIG. 1

T128 polypeptide amino acid sequence.

FIG. 2

Nucleic acid sequence encoding T128. Start codon (ATG) and stop codon (TGA) are underlined. The nucleic acid sequence encoding T128 is also provided in SEQ. ID No. 2, the corresponding open reading frame is provided in SEQ. ID No. 3 and the 25 nucleotide fragment given in bold is provided in SEQ. ID No. 4.

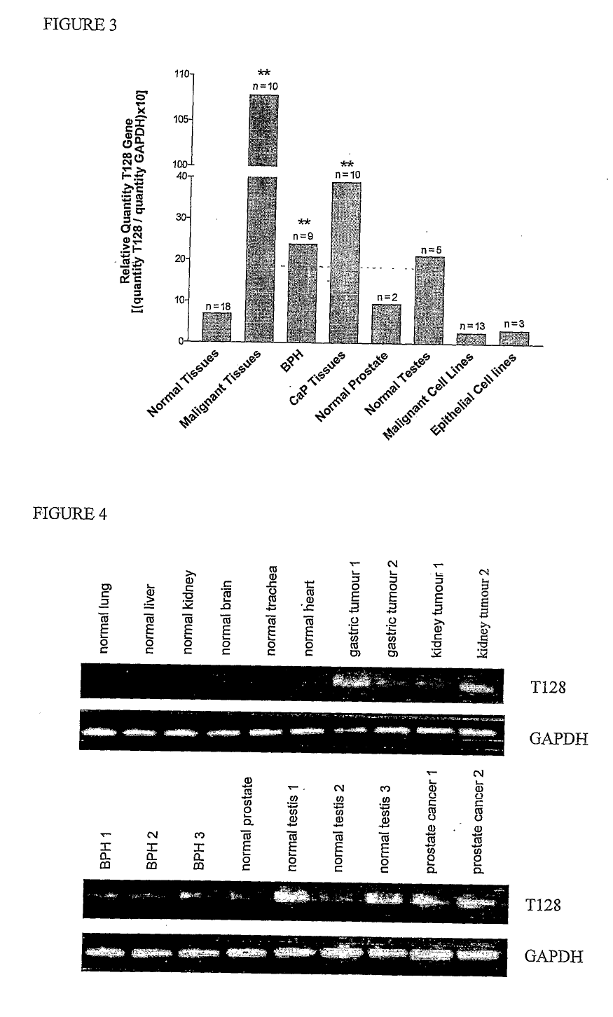

FIG. 3

Tissue expression analysis of T128 transcripts analysed by RT-Q-PCR. To calculate an arbitrary level of expression of T128 using RT-Q-PCR a standard curve was generated using serial dilutions of testis cDNA as template and assigning the dilutions arbitrary concentration values. T128 gene specific primers were then used in the RT-Q-PCR reactions to generate gene specific product. A panel of normal and malignant samples was run in parallel to the standard curve and arbitrary quantities of T128 expression were calculated from the standard curve. The same cDNA samples were tested for GAPDH, an internal control. These expression levels were then normalised to GAPDH by dividing the quantity of T128 gene specific product by the quantity of GAPDH in the same sample. T128 transcripts are over expressed in malignant tissues (gastric and kidney) (p<0.05) and normal testes (p<0.01) when compared to normal tissues (lung, liver, heat, brain, trachea, kidney, adrenal gland, endometrium, colon, breast, PBMC, tonsil, small intestine, vagina, muscle, placenta and ovary).

FIG. 4

Expression of T128 analysed by RT-PCR. Varying levels of T128 expression are also found in selected BPH samples. The same cDNA samples were tested for GAPDH, as an internal control.

Technique Used to Identify Genes Encoding Tumour Antigens (SEREX Technique)

The technique for the expression of cDNA libraries from human testes is described and was performed according to published methodology (Sahin et. al. Proc Natl. Acad. Sci. 92, 11810-11813, 1995).

SEREX has been used to analyze gene expression in tumour tissues from human melanoma, renal cell cancer, astrocytoma, oesophageal squamous cell carcinoma, colon cancer, lung cancer and Hodgkin's disease. Sequence analysis revealed that several different antigens, including HOM-MEL-40, HOM-HD-397, HOM-RCC-1.14, NY-ESO-1, NY-LU-12, NY-CO-13 and MAGE genes, were expressed in these malignancies, demonstrating that several human tumour types express multiple antigens capable of eliciting an immune response in the autologous host. This represents an alternative and more efficient approach to identify tumour markers, and offers distinct advantages over previously used techniques:
1) the use of fresh tumour specimens to produce the cDNA libraries obviates the need to culture tumour cells in vitro and therefore circumvents artefacts, such as loss or neo-antigen expression and genetic and phenotypic diversity generated by extended culture;
2) the analysis is restricted to antigen-encoding genes expressed by the tumour in vivo;
3) using cDNA expression cloning, the serological analysis (in contrast to autologous typing) is not restricted to cell surface antigens, but covers a more extensive repertoire of cancer-associated proteins (cytosolic, nuclear, membrane, etc.);
4) in contrast to techniques using monoclonal antibodies, SEREX uses poly-specific sera to scrutinise single antigens that are highly enriched in lytic bacterial plaques allowing the efficient molecular identification of antigens following sequencing of the cDNA. Subsequently the tissue-expression spectrum of the antigen can be determined by the analysis of the mRNA expression patterns using, for example, northern blotting and reverse transcription-PCR (RT-PCR), on fresh normal and malignant (autologous and allogeneic) tissues. Likewise, the prevalence of antibody in cohorts of cancer patients and normal controls can be determined.

The T128 sequence can be isolated by the polymerase chain reaction using the following pair of primers:

| | |
|---|---|
| T128 Primer 1: | |
| GAGAGAGCGATCAAGAGAAAGG | (SEQ ID No. 5) |
| T128 Primer 2: | |
| ATCTCTGTGCCGCCTATCAT | (SEQ ID No. 6) |

under the following reaction conditions:

| Temperature (° C.) | Time (minutes) | Cycles | Comment |
|---|---|---|---|
| 95 | 15 | 1 | Denaturation |
| 95 | 0.75 | 33 | Denaturation |
| 58 | 0.75 | | Annealing |
| 72 | 1.5 | | Extension |
| 95 | 1 | 1 | Denaturation |

The terms ‘denaturation’, ‘annealing’ and ‘extension’ are well-known and understood to the person skilled in the art of PCR and the reader is directed to ‘FIG. 10.1 in Principles of Gene Manipulation. An Introduction to Genetic Engineering (5th Edition, 1994). R. W. Old and S. B. Primrose (Publisher: Blackwell Science).
Extraction of Nucleic Acid from Tissue

A prokaryotically expressed cDNA library can be constructed by isolating 10 μg of total RNA from normal testes tissues, treating the total RNA with Calf Intestinal Phosphatase to remove 5′-phosphates from un-capped RNAs, removing the cap structure from fill-length mRNA by Tobacco Acid Pyrophosphatase (TAP) and ligating RNA adapters to mRNA molecules containing 5′phosphate. The actual library used was a commercially available λTriplEx2™ Human testes large-insert cDNA library (Product 634220, Clontech, Palo Alto, Calif., USA).

Serological Analysis of Recombinant cDNA Expression Libraries (SEREX)

The SEREX approach allows an unbiased search for an antibody response and the direct molecular definition of immunogenic tumour proteins based on their reactivity with allogeneic patient sera. In this approach, a prokaryotically expressed cDNA library constructed from normal human testes was immunoscreened with absorbed and diluted patients' sera for the detection of tumour antigens that have elicited a high-titer immunoglobulin (Ig) G humoral response. Such a humoral response implies T-cell recognition of the detected antigens by helper T cells. Thus, even though the antigens are initially identified by antibodies, the method reveals tumour products that can then be analysed in the context of cell-mediated immunity. The SEREX approach can then be modified and used to determine the reactivity of identified antigens with panels of human sera including prostate cancer patient sera and normal donor sera.

In this case, the SEREX approach was modified by pooling allogeneic sera from four prostate cancer patients to screen a normal testes cDNA library, rather than a cancer cDNA library, to identify cancer-testes (C-T) antigens.

Inserts were sequenced on a ABI Prism semi-automated sequencer using T7 primers specific for the vector.

Rapid Amplification of the cDNA Ends (RACE)

Sequencing of the clones identified following immunoscreening allows only the cDNA insert sequence to be in attained full. The complete 5′- and 3′-ends of the sequence can then be obtained using a procedure termed rapid amplification of cDNA ends (RACE). The SMART™ RACE cDNA amplification kit (BD Biosciences, Clontech, Palo Alto, Calif., USA) used provides a novel method for performing both 5′- and 3′-RACE. In brief, first-strand cDNA synthesis is performed on high quality testes RNA expressing the gene of interest. The cDNA for 5′-RACE-ready cDNA is synthesised using a modified lock-docking oligo (dT) primer and the SMART II oligo. The 3′-RACE-ready cDNA is synthesised using a traditional reverse transcription procedure but with a special oligo (dT) primer. This primer also has a portion of the SMART sequence at its 5′ end. By incorporating the SMART sequence into both the 5′- and 3′-RACE-ready cDNA populations, both PCR reactions can be primed using the universal primer mix (UPM) A, that recognises the SMART sequence, in conjunction with distinct gene-specific primers designed to amplify either in the 5′ or 3′ direction.

Setting up 5′ and 3′ RACE PCR reactions

| | | |
|---|---|---|
| | 5′-RACE | 3′-RACE |
| Component | sample | sample |

| | | | | |
|---|---|---|---|---|
| RACE-Ready cDNA | 2.5 | μl | 2.5 | μl |
| UPM (10X) | 5 | μl | 5 | μl |
| 5′ RACE gene specific primer | 1 | μl | | |
| (SEQ ID No 7) | | | | |
| TCTTCCTTGACTGCCACTTCGACTTCGT | | | | |
| 3′ RACE gene specific primer | | | 1 | μl |
| (SEQ ID No 8) | | | | |
| CGGCCAAGATCCCGAAGTATTGACC | | | | |
| Master Mix | 41.5 | μl | 41.5 | μl |

A primary PCE was carried out using the 3′RACE gene specific primers under the following reaction conditions:

| Temperature (° C.) | Time (minutes) | Cycles | Comment |
|---|---|---|---|
| 94 | 0.5 | 5 | Denaturation |
| 72 | 2 | | Extension |
| 94 | 0.5 | 35 | Denaturation |
| 70 | 0.5 | | Annealing |
| 72 | 2 | | Extension |

Subsequently the reaction product from the primary 3′ RACE PCR was used as template nucleic acid in 3′ RACE nested PCR using 3′ RACE nested PCR primers under the following conditions:

| Temperature (° C.) | Time (minutes) | Cycles | Comment |
|---|---|---|---|
| 94 | 0.5 | 4 | Denaturation |
| 70 | 0.5 | | Annealing |
| 72 | 3 | | Extension |

| | |
|---|---|
| 3′ RACE gene specific nested primer: | |
| ACGAAGTCGAAGTGGCAGTCAAGGAAGA. | (SEQ ID No 9) |

A primary PCR was carried out using the 5′RACE gene specific primers under the following reaction conditions:

| Temperature (° C.) | Time (minutes) | Cycles | Comment |
|---|---|---|---|
| 94 | 0.5 | 5 | Denaturation |
| 72 | 2 | | Extension |
| 94 | 0.5 | 5 | Denaturation |
| 70 | 0.5 | | Annealing |
| 72 | 2 | | Extension |
| 94 | 0.5 | 30 | Denaturation |
| 68 | 0.5 | | Annealing |
| 72 | 2 | | Extension |

An equivalent ‘nested’ PCR reaction was not undertaken for the 5′RACE gene since the primary 5′RACE PCR generated a sufficiently pure product and sequencing of this product demonstrated that the correct product had been generated.

The reactivity of promising positive clones against patient and normal donor serum was determined by immunoscreening the clones against and allogeneic panel of 10 prostate cancer patients' sera and 10 healthy donor patients' sera. The methodology used was the same as the SEREX methodology (described above) with the following modifications: The positive clone (T128) and a negative clone (blue phage) were plated out on a small LB agar plate at a ratio of 1:2 to give a titre of approximately 600 pfu/plate. The steps for detection of false positives, subcloning and re-testing were not necessary. Following expression overnight, plaque expression and transferral of the plaques to nitrocellulose membranes, the nitrocellulose membranes were incubated with the individual serum and colour developed as described previously. The results would then be recorded simply as being positive (plaques visible) or negative (no plaques visible).

Quantitiative Real Time Reverse Transcription Polymerase Chain Reaction (RT-Q-PCR)

RT-Q-PCR was used to determine the tissue specificity of T128 expression of SEREX-defined genes in various tissues and cell lines. The indicator dye used was SYBR green.

RT-Q-PCR quantitates the initial amount of template most specifically, sensitively and reproducibly, and is a preferable alternative to semi-quantitative RT-PCR which detects the amount of final amplified product. Real time PCR monitors the fluorescence emitted during the reaction as an indicator of amplicon production during each PCR cycle as opposed to the endpoint detection by conventional PCR methods. The quantitative detection of the amplicon can be measured using a DNA-binding agent called SYBR green (Molecular Probes inc., Eugene, Oreg., USA). SYBR green is a non-sequence specific fluorescent intercalating agent that only binds to double stranded DNA within the minor groove.

The Mx4000 apparatus measures the fluorescence of each sample at the end of the annealing step, and at the end of each cycle when creating the dissociation curve. After the RT-PCR reaction, the software program plots linear values of fluorescence (dRn) against cycle number. After background adjustment, the Ct value, which is defined as the number of cycles at which the reaction crosses a threshold value, i.e. the fluorescence due to the RT-PCR product exceeds the background level, is calculated for each sample by the software. The software produces a standard curve by measuring the Ct value of each standard and plotting it against the approximate concentrations for the corresponding standard dilution. The expression level of the unknown genes in a given RNA sample are then normalised to the housekeeping gene GAPDH. The normalised expression of each gene is calculated by dividing the Ct value for the unknown gene in a sample by the Ct value for GAPDH in the same sample. Thus, a sample with high level expression of a gene will have a lower Ct value because the gene is more abundant, hence it takes less cycles for the fluorescence to exceed that of background levels. Therefore, when calculating the normalised expression for that gene the Ct value would be lower than a gene that is less abundant. This should be remembered when observing the normalised expression graphs because the lower the Ct value the more abundant the gene is in the sample. Derivation of this fraction is independent of RNA sample concentration, eliminating the requirement to measure RNA concentration accurately.

The RT-Q-PCR reactions were performed in the Mx4000 QPCR system (Stratagene, UK) using SYBR green fluorescent dye (Yin, J. L. et al., 2001. Immuno. Cell Biol. 79(3):213-21). RNA samples were DNase treated in order to remove genomic DNA following standard protocols. Thermocycling for each reaction was done in a final volume of 25 μl containing 1 μl of template (1:10 diluted), or standard, 12.5 μl SYBR green master mix (Qiagen, UK) containing Hot Start®Taq DNA polymerase, reaction buffer, ROX reference dye, SYBR green dye, magnesium chloride and deoxynucleotides, and pre-optimised amounts of gene-specific forward and reverse primers. This was then made up to 25 μl with Qiagen water. In each experiment a minimum of 8 no-template controls should be included to ensure no contamination has occurred and also to indicate the degree of amplification due to primer dimers. Also included were 4 RT-negative (no reverse transcription) samples to ensure that genomic DNA had been completely removed following DNase treatment.

Gene specific primer sequences SEQ ID No 8 and 9 (see above) were used in both conventional and real time PCR.

In Silico Analysis of T128

The amino acid sequence of T128 was analysed using search programs including PROSITE (accessible at www.expasy.ch/prosite/), PSORT (accessible at http://psort.nibb.ac.jp) and Pfam (accessible at http://www.sanger.ac.uk/cgi-bin/Pfam/nph-search.cgi). These identified:
Chromosomal locallization Ip13.3
N-glycosylation site at position 264-267
cAMP-phosphorylation sites 60-63, 118-121, 130-133, 219-222, 226-229, 301-304, 327-330.
PKC phosphorylation sites at positions 65-67, 71-73, 75-77, 222-224, 259-261, 270-272, 288-290, 300-302, 310-312, 322-324.
Casein kinase 2 phosphorylation sites at positions 2-5, 12-15, 92-95, 197-200, 222-225.
Tyrosine phosphorylation sites at position 167-173.
Amidation sites at positions 165-168, 247-250.
Arginine rich region 56-254.
Lysine rich region 204-322.
Senine rich region 229-322.

### RESULTS

Tissue Specificity

The results of tissue specificity studies are shown in FIGS. 3 and 4. The Table shows the reactivity of patients sera with T128.

Tables of results to show the immunoreactivity of 10 individual prostate cancer patients' sera and 10 individual normal patients' sera with the 6 positive clones identified following immunoscreening:

Immunoreactivity of Prostate Cancer Patients' Serum

| | Immunoreactivity Observed with Serum | | | | | | | | | |
|---|---|---|---|---|---|---|---|---|---|---|
| Clone | Pr27 | Pr29 | Pr32 | Pr33 | Pr34 | Pr36 | Pr37 | Pr38 | Pr39 | Pr48 |
| 128 | + | − | + | + | + | − | + | + | − | − |

Immunoreactivity of Normal Patients' Serum

| | Immunoreactivity Observed with Serum | | | | | | | | | |
|---|---|---|---|---|---|---|---|---|---|---|
| Clone | Nor1 | Nor2 | Nor3 | Nor5 | Nor12 | Nor13 | Nor14 | Nor19 | Nor20 | Nor21 |
| 128 | − | − | − | + | − | − | + | + | − | − |

The antigen is testes specific in normal tissues, but is highly expressed in cancerous tissues.
Sequence

T128 was sequenced and the homology of sequence was compared with homologous sequences on Genbank. This showed some overlap with a hypothetical protein, FLJ10330.

The sequence of T128 is shown in FIGS. 1 and 2.

### CONCLUSIONS

T128 is an antigen that is testes specific in normal tissue. Cancerous tissue has high levels of expression of this antigen from cancers derived from tissues other than testes. Prostate, kidney and gastric cancers have high levels of the antigen expressed, making it a good marker for cancer and a target as a therapeutic agent. The immunogenic activity of this antigen is demonstrated by the identification of antibodies in cancer sufferers which assisted in identifying this antigen via the use of the SEREX method.

1. An isolated nucleic acid molecule selected from the group consisting of:
(a) nucleic acid molecules encoding T128 polypeptide (SEQ ID NO: 1), a polypeptide at least 80% identical to SEQ ID NO: 1, or a fragment thereof, which is capable of cross-reacting with sera from patients with prostate cancer;
(b) nucleic acid molecules comprising the nucleotide sequence between nucleic acid residues 642 and 1688 of SEQ ID NO: 2;
(c) nucleic acid molecules, the complementary strand of which specifically hybridises to a nucleic acid molecule in (a) or (b); and
(d) nucleic acid molecules the sequence of which differs from the sequence of the nucleic acid molecule in (a), (b), or (c) due to the degeneracy of the genetic code.

2. An isolated nucleic acid molecule according to claim 1, encoding the polypeptide sequence of SEQ ID NO: 1.

3. An isolated nucleic acid molecule which is at least 80% homologous to the nucleic acid molecule according to claim 1 and which encodes a polypeptide which is expressed in higher concentrations in cancerous tissue compared to that tissue when in a normal state.

4. An isolated nucleic acid molecule comprising at least 15 nucleic acids capable of specifically hybridising to a sequence within a nucleic acid molecule according to claim 1.

5. A vector comprising a nucleic acid molecule according to claim 1.

6. A host cell comprising a vector according to claim 5.

7. An isolated protein comprising an amino acid sequence encoded by a nucleic acid molecule according to claim 1.

8. An isolated protein according to claim 7 which comprises the amino acid sequence of SEQ ID NO: 1.

9. A fragment or derivative of the protein according to claim 7.

10. A monoclonal antibody capable of specifically binding to the protein of claim 7, or a fragment or derivative thereof.

11-12. (canceled)

13. A method of detecting or monitoring cancer in a patient comprising the step of detecting or monitoring elevated levels of a nucleic acid molecule comprising the nucleic acid molecule according to claim 1 in a sample from the patient.

14. A method of detecting or monitoring cancer comprising the step of detecting or monitoring elevated levels of a nucleic acid molecule comprising the nucleic acid molecule according to claim 1 in combination with a reverse transcription polymerase chain reaction.

15. A method of detecting or monitoring cancer comprising the step of detecting or monitoring elevated levels of the protein according to claim 7.

16. The method according to claim 15 wherein the detecting or monitoring step includes a monoclonal antibody selective for and capable of detecting the protein.

17. The method according to claim 16 wherein the detecting or monitoring step includes an Enzyme-Linked ImmunoSorbant Assay.

18. The method according to claim 13, wherein the cancer is a gastro-intestinal cancer, kidney cancer or a prostate cancer.

19. A kit comprising the nucleic acid molecule as defined in claim 1 for use with a method of detecting or monitoring cancer.

20. A method of prophylaxis or treatment of cancer comprising the step of administering to a patient a pharmaceutically effective amount of a nucleic acid molecule comprising the nucleic acid molecule according to claim 1 or a pharmaceutically effective fragment thereof.

21. A method of prophylaxis or treatment of cancer comprising the step of administering to a patient a pharmaceutically effective amount of a nucleic acid molecule hybridisable under high stringency conditions to a nucleic acid molecule comprising the nucleic acid molecule according to claim 1 or a pharmaceutically effective fragment thereof.

22. A method of prophylaxis or treatment of cancer comprising the step of administering to a patient a pharmaceutically effective amount of a the protein according to claim 7 or a pharmaceutically effective fragment thereof.

23. A method of prophylaxis or treatment of cancer comprising the step of administering to a patient a pharmaceutically effective amount of the monoclonal antibody according to claim 10.

24. The method according to claim 20, wherein the cancer is a gastro-intestinal cancer.

25. A vaccine comprising a nucleic acid molecule comprising the nucleic acid molecule according to claim 1 or a pharmaceutically effective fragment thereof, and a pharmaceutically acceptable carrier.

26. A vaccine comprising the protein according to claim 7 or a pharmaceutically effective fragment thereof, and a pharmaceutically acceptable carrier.

27. The polypeptide according to claim 7 or a pharmaceutically effective fragment thereof, attached to a carrier protein.

28. A kit comprising the protein according to claim 7 for use with a method of detecting or monitoring cancer.

29. A kit comprising the monoclonal antibody according to claim 10 for use with a method of detecting or monitoring cancer.

30. An immunogenic composition comprising a nucleic acid molecule comprising the nucleic acid molecule according to claim 1 or a pharmaceutically effective fragment thereof, and a pharmaceutically acceptable carrier.

31. An immunogenic composition comprising the protein according to claim 7 or a pharmaceutically effective fragment thereof, and a pharmaceutically acceptable carrier.
